(19) United States
(12) Patent Application Publication
(10) Pub. No.: US 2023/0000584 A1
(43) Pub. Date: Jan. 5, 2023

(54) SYSTEMS AND METHODS FOR AIDING NON-CONTACT DETECTOR PLACEMENT IN NON-CONTACT PATIENT MONITORING SYSTEMS

(71) Applicant: Covidien LP, Mansfield, MA (US)

(72) Inventors: Dominique D. JACQUEL, Edinburgh (GB); Dean MONTGOMERY, Edinburgh (GB); Philip C. SMIT, Edinburgh (GB); Paul S. ADDISON, Edinburgh (GB)

(21) Appl. No.: 17/662,055

(22) Filed: May 4, 2022

Related U.S. Application Data
(60) Provisional application No. 63/216,698, filed on Jun. 30, 2021.
(60) Provisional application No. 63/268,551, filed on Feb. 25, 2022.

(51) Int. Cl. A61B 90/00

(52) U.S. Cl. CPC A61B 90/36; A61B 2090/365

(57) ABSTRACT

Systems and methods for aiding a clinician in the proper positioning, placing or otherwise locating of a non-contact detector component of a non-contact patient monitoring system are described. The systems and methods may employ a targeting aid superimposed on a display screen component of the non-contact patient monitoring system, the targeting aid being designed to assist the clinician in properly locating the non-contact detector for proper and accurate functioning of the non-contact patient monitoring system. The systems and methods described herein may also employ a bendable mounting arm to which the non-contact detector is attached such that the non-contact detector can be easily moved into the proper location when used in conjunction with the targeting aid superimposed on the display.

### CROSS-REFERENCE TO RELATED APPLICATIONS

The present application claims benefit of priority to U.S. Provisional Patent Application No. 63/216,698, entitled “SYSTEMS AND METHODS FOR AIDING NON-CONTACT DETECTOR PLACEMENT IN NON-CONTACT PATIENT MONITORING SYSTEMS,” filed on Jun. 30, 2021, and U.S. Provisional Patent Application No. 63/268,551, entitled “SYSTEMS AND METHODS FOR AIDING NON-CONTACT DETECTOR PLACEMENT IN NON-CONTACT PATIENT MONITORING SYSTEMS,” filed on Feb. 25, 2022, which are specifically incorporated by reference herein for all that they disclose or teach.

### TECHNICAL FIELD

The present disclosure relates to systems and methods for aiding a clinician in the proper positioning, placing or otherwise locating of a non-contact detector component of a non-contact patient monitoring system. While generally not limited to any specific equipment, the non-contact detector component of the non-contact patient monitoring system can be a camera, such as a depth sensing camera. In some embodiments, the systems and methods described herein employ a targeting aid superimposed on a display component of the non-contact patient monitoring system, the targeting aid being designed to assist the clinician in properly locating the non-contact detector for proper and accurate functioning of the non-contact patient monitoring system. The systems and methods described herein may also employ a bendable mounting arm to which the non-contact detector is attached such that the non-contact detector can be easily moved into the proper location when used in conjunction with the targeting aid superimposed on the display. In some embodiments, the attachment mechanism between the bendable arm and the non-contact detector includes a gimbal such that the non-contact detector remains aligned in the correct direction (e.g., lens aligned generally horizontally) regardless of the manner in which the bendable arm is moved or manipulated.

### BACKGROUND

A variety of technologies have been developed for non-contact patient monitoring. Some of these technologies employ depth sensing technologies, which can be employed to determine a number of physiological and contextual parameters, including, but not limited to, respiration rate, tidal volume, minute volume, effort to breath, patient activity, presence in bed, etc. By way of example, U.S. Pat. Nos. 10,702,188 and 10,939,824 and U.S. Published Patent Application Nos. 2019/0209046, 2019/0380599, 2019/0380807, and 2020/0046302, each of which is incorporated herein by reference in its entirety, describe various non-contact patient monitoring technologies employing depth sensing technology for determining patient physiological and contextual parameters.

The efficacy and usefulness of such non-contact patient monitoring system may depend at least in part on the correct positioning of the depth sensing camera used as part of the non-contact patient monitoring system. Correct positioning of the depth sensing camera may include, for example, both the location of the camera relative to the patient being monitored and the camera's distance away from the patient. If either or both parameters are not properly set, the data obtained from the depth sensing camera may be inconsistent and/or inaccurate. In contrast, when the camera is properly positioned, the ability to localize the visualization on the screen to the patient's targeted region is greatly improved. This, in turn, greatly improves the ability to generate accurate and reliable data that is subsequently used to derive various patient parameters.

It has been found through experimentation and clinical trials that clinicians often improperly locate the depth sensing camera, thus leading to diminished reliability and quality in patient parameters derived from non-contact patient monitoring systems. Accordingly, a need exists for methods and systems that aid the clinician in proper location of a depth sensing component of a non-contact patient monitoring system.

### SUMMARY

Described herein are various embodiments of methods and systems for aiding non-contact detector placement in a non-contact patient monitoring system.

In one embodiment, a video-based patient monitoring method includes: obtaining from a non-contact detector a video signal, the video signal encompassing at least a torso region of a patient; displaying on a display screen a video based on the video signal; superimposing a target over a portion of the video displayed on the display screen; and providing an indication that the torso region of the patient in the displayed video is located within the target.

In another embodiment, a video-based patient monitoring method includes: displaying on a display screen a patient image based on a video signal obtained from a video camera, the patient image having superimposed thereon a target encompassing a portion of the patient image; and manipulating a bendable arm to which the video camera is attached to reposition the video camera until a patient target area in the patient image is located within the target on the display screen; wherein a connection between the bendable arm and the video camera includes a gimbal, the gimbal being configured to maintain the orientation of the video camera in a position generally parallel to a bed on which the patient is positioned during manipulation of the bendable arm.

In another embodiments, a video-based patient monitoring system includes: a video camera configured to obtain a video signal; a bendable arm attached at a distal end to the video camera; and a display, the display configured to display a patient image based on the video signal and superimpose over the patient image a target; wherein the video camera is moveable about a patient via manipulation of the bendable arm; and wherein the system is configured to: automatically determine when a target patient area of the patient image is located within the target superimposed on the patient image via manipulation of the bendable arm and corresponding repositioning of the video camera; and provide visual and/or audible feedback when the system automatically determines that the target patient area of the patient image is located within the target superimposed on the patient image.

In another embodiment, a video-based patient monitoring method includes projecting a target onto a patient using a projector connected to a non-contact detector, manipulating the position of the non-contact detector until a portion of the patient's body is located within the target, obtaining from the non-contact detector a video signal, the video signal encompassing at least the portion of the patient's body located within the target, and displaying on a display screen a video based on the video signal. In some embodiments, the display screen is located remote (e.g., not visible from) the non-contact detector.

In another embodiment, a video-based patient monitoring system includes a video camera configured to obtain a video signal, a bendable arm attached at a distal end to the video camera, a projector connected to the video camera, the projector being configured to project a target on to a patient when the patient is in a field of view of the video camera, and a display located remote from the video camera and the projector, the display being configured to display a patient image based on the video signal. The video camera is moveable about the patient via manipulation of the bendable arm, and the projector is connected to the video camera in a manner that ensures that when the patient's torso is located within the projected target, the video camera can be used to obtain depth data suitable for use in calculating a patient breathing parameter.

### BRIEF DESCRIPTION OF THE DRAWINGS

Many aspects of the present disclosure can be better understood with reference to the following drawings. The components in the drawing are not necessarily to scale. Instead, emphasis is placed on illustrating clearly the principles of the present disclosure. The drawings should not be taken to limit the disclosure to the specific embodiments depicted but are for explanation and understanding only.

FIG. 1 is a schematic view of a video-based patient monitoring system configured in accordance with various embodiments of the present technology.

FIG. 2 is a block diagram illustrating a video-based patient monitoring system having a computing device, a server, and one or more image capturing devices, and configured in accordance with various embodiments of the present technology.

FIG. 3A is an illustration of components of a video-based patient monitoring system configured in accordance with various embodiments of the present technology.

FIG. 3B is an illustration of components of a video-based patient monitoring system configured in accordance with various embodiments of the present technology, the video-based patient monitoring system being installed proximate a patient bed.

FIG. 3C is an illustration of components of a video-based patient monitoring system configured in accordance with various embodiments of the present technology.

FIG. 4 is an illustration of a display of a video-based patient monitoring system configured in accordance with various embodiments of the present technology.

FIG. 5 is an illustration of various states of a display of a video-based patient monitoring system based on different camera locations, the display and system configured in accordance with various embodiments of the present technology.

FIG. 6 is an illustration of various states of a display of a video-based patient monitoring system based on different patient image orientations, the display and system configured in accordance with various embodiments of the present technology.

FIG. 7 is an illustration of a display of a video-based patient monitoring system configured in accordance with various embodiments of the present technology.

FIG. 8 is an illustration of a display of a video-based patient monitoring system configured in accordance with various embodiments of the present technology.

FIG. 9 is a flow chart of a method for video-based non-contact patient monitoring configured in accordance with various embodiments of the present technology.

FIG. 10 is an illustration of components of a video-based patient monitoring system configured in accordance with various embodiments of the present technology.

FIG. 11 is an illustration of various targets projected on a patient in accordance with various embodiments of the present technology.

FIG. 12 is a flow chart of a method for video-based non-contact patient monitoring configured in accordance with various embodiments of the present technology

### DETAILED DESCRIPTION

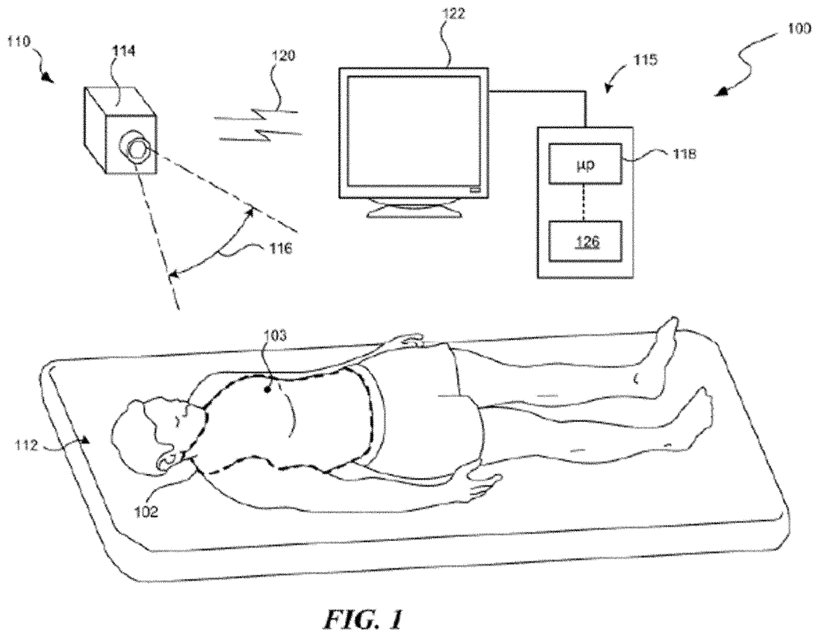

Specific details of several embodiment of the present technology are described herein with reference to FIGS. 1-12. Although many of the embodiments are described with respect to devices, systems, and methods for aiding non-contact detectors placement in non-contact patient monitoring technology, other applications and other embodiments in addition to those described herein are within the scope of the present technology. For example, at least some embodiments of the present technology can be useful for video-based monitoring of non-patients (e.g., elderly or neonatal individuals within their homes). It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a schematic view of a patient 112 and a video-based patient monitoring system 100 configured in accordance with various embodiments of the present technology. The system 100 includes a non-contact detector 110 and a computing device 115. In some embodiments, the detector 110 can include one or more image capture devices, such as one or more video cameras. In the illustrated embodiment, the non-contact detector 110 includes a video camera 114. The non-contact detector 110 of the system 100 is placed remote from the patient 112. More specifically, the video camera 114 of the non-contact detector 110 is positioned remote from the patient 112 in that it is spaced apart from and does not contact the patient 112. The camera 114 includes a detector exposed to a field of view (FOV) 116 that encompasses at least a portion of the patient 112.

The camera 114 can capture a sequence of images over time. The camera 114 can be a depth sensing camera, such as a Kinect camera from Microsoft Corp. (Redmond, Wash.) or Intel camera such as the D415, D435, and SR305 cameras from Intel Corp, (Santa Clara, Calif.). A depth sensing camera can detect a distance between the camera and objects within its field of view. Such information can be used to determine that a patient 112 is within the FOV 116 of the camera 114 and/or to determine one or more regions of interest (ROI) to monitor on the patient 112. Once a ROI is identified, the ROI can be monitored over time, and the changes in depth of regions (e.g., pixels) within the ROI 102 can represent movements of the patient 112.

In some embodiments, the system 100 determines a skeleton-like outline of the patient 112 to identify a point or points from which to extrapolate a ROI. For example, a skeleton-like outline can be used to find a center point of a chest, shoulder points, waist points, and/or any other points on a body of the patient 112. These points can be used to determine one or more ROIs. For example, a ROI 102 can be defined by filling in area around a center point 103 of the chest, as shown in FIG. 1. Certain determined points can define an outer edge of the ROI 102, such as shoulder points. In other embodiments, instead of using a skeleton, other points are used to establish a ROI. For example, a face can be recognized, and a chest area inferred in proportion and spatial relation to the face. In other embodiments, a reference point of a patient's chest can be obtained (e.g., through a previous 3-D scan of the patient), and the reference point can be registered with a current 3-D scan of the patient. In these and other embodiments, the system 100 can define a ROI around a point using parts of the patient 112 that are within a range of depths from the camera 114. In other words, once the system 100 determines a point from which to extrapolate a ROI, the system 100 can utilize depth information from the depth sensing camera 114 to fill out the ROI. For example, if the point 103 on the chest is selected, parts of the patient 112 around the point 103 that are a similar depth from the camera 114 as the point 103 are used to determine the ROI 102.

In another example, the patient 112 can wear specially configured clothing (not shown) that includes one or more features to indicate points on the body of the patient 112, such as the patient's shoulders and/or the center of the patient's chest. The one or more features can include visually encoded message (e.g., bar code, QR code, etc.), and/or brightly colored shapes that contrast with the rest of the patient's clothing. In these and other embodiments, the one or more features can include one or more sensors that are configured to indicate their positions by transmitting light or other information to the camera 114. In these and still other embodiments, the one or more features can include a grid or another identifiable pattern to aid the system 100 in recognizing the patient 112 and/or the patient's movement. In some embodiments, the one or more features can be stuck on the clothing using a fastening mechanism such as adhesive, a pin, etc. For example, a small sticker can be placed on a patient's shoulders and/or on the center of the patient's chest that can be easily identified within an image captured by the camera 114. The system 100 can recognize the one or more features on the patient's clothing to identify specific points on the body of the patient 112. In turn, the system 100 can use these points to recognize the patient 112 and/or to define a ROI.

In some embodiments, the system 100 can receive user input to identify a starting point for defining a ROI. For example, an image can be reproduced on a display 122 of the system 100, allowing a user of the system 100 to select a patient 112 for monitoring (which can be helpful where multiple objects are within the FOV 116 of the camera 114) and/or allowing the user to select a point on the patient 112 from which a ROI can be determined (such as the point 103 on the chest of the patient 112). In other embodiments, other methods for identifying a patient 112, identifying points on the patient 112, and/or defining one or more ROI's can be used.

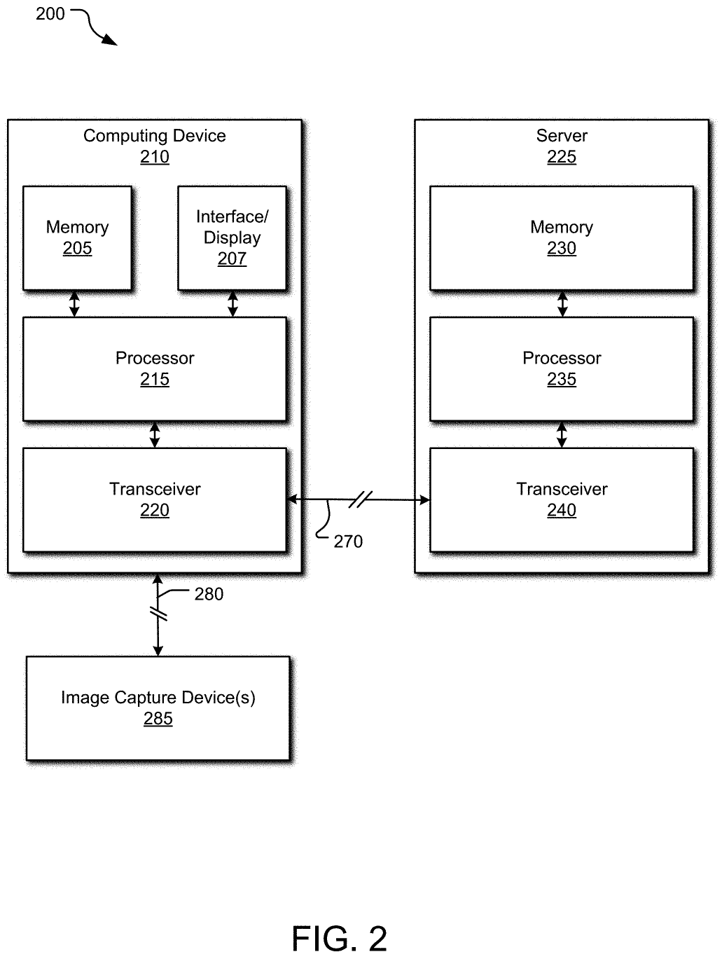

The images detected by the camera 114 can be sent to the computing device 115 through a wired or wireless connection 120. The computing device 115 can include a processor 118 (e.g., a microprocessor), the display 122, and/or hardware memory 126 for storing software and computer instructions. Sequential image frames of the patient 112 are recorded by the video camera 114 and sent to the processor 118 for analysis. The display 122 can be remote from the camera 114, such as a video screen positioned separately from the processor 118 and the memory 126. Other embodiments of the computing device 115 can have different, fewer, or additional components than shown in FIG. 1. In some embodiments, the computing device 115 can be a server. In other embodiments, the computing device 115 of FIG. 1 can be additionally connected to a server (e.g., as shown in FIG. 2 and discussed in greater detail below). The captured images/video can be processed or analyzed at the computing device 115 and/or a server to determine, e.g., a patient's position while lying in bed or a patient's change from a first position to second position while lying in bed. In some embodiments, some or all of the processing may be performed by the camera, such as by a processor integrated into the camera or when some or all of the computing device 115 is incorporated into the camera.

FIG. 2 is a block diagram illustrating a video-based patient monitoring system 200 (e.g., the video-based patient monitoring system 100 shown in FIG. 1) having a computing device 210, a server 225, and one or more image capture devices 285, and configured in accordance with various embodiments of the present technology. In various embodiments, fewer, additional, and/or different components can be used in the system 200. The computing device 210 includes a processor 215 that is coupled to a memory 205. The processor 215 can store and recall data and applications in the memory 205, including applications that process information and send commands/signals according to any of the methods disclosed herein. The processor 215 can also (i) display objects, applications, data, etc. on an interface/display 207 and/or (ii) receive inputs through the interface/display 207. As shown, the processor 215 is also coupled to a transceiver 220.

The computing device 210 can communicate with other devices, such as the server 225 and/or the image capture device(s) 285 via (e.g., wired or wireless) connections 270 and/or 280, respectively. For example, the computing device 210 can send to the server 225 information determined about a patient from images captured by the image capture device(s) 285. The computing device 210 can be the computing device 115 of FIG. 1. Accordingly, the computing device 210 can be located remotely from the image capture device(s) 285, or it can be local and close to the image capture device(s) 285 (e.g., in the same room). In various embodiments disclosed herein, the processor 215 of the computing device 210 can perform the steps disclosed herein. In other embodiments, the steps can be performed on a processor 235 of the server 225. In some embodiments, the various steps and methods disclosed herein can be performed by both of the processors 215 and 235. In some embodiments, certain steps can be performed by the processor 215 while others are performed by the processor 235. In some embodiments, information determined by the processor 215 can be sent to the server 225 for storage and/or further processing.

In some embodiments, the image capture device(s) 285 are remote sensing device(s), such as depth sensing video camera(s), as described above with respect to FIG. 1. In some embodiments, the image capture device(s) 285 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter wave scanning technology can be utilized to scan a patient, which can be used to define and/or monitor a ROI. Advantageously, such technologies can be able to “see” through clothing, bedding, or other materials while giving an accurate representation of the patient's skin. This can allow for more accurate measurements, particularly if the patient is wearing baggy clothing or is under bedding. The image capture device(s) 285 can be described as local because they are relatively close in proximity to a patient such that at least a part of a patient is within the field of view of the image capture device(s) 285. In some embodiments, the image capture device(s) 285 can be adjustable to ensure that the patient is captured in the field of view. For example, the image capture device(s) 285 can be physically movable, can have a changeable orientation (such as by rotating or panning), and/or can be capable of changing a focus, zoom, or other characteristic to allow the image capture device(s) 285 to adequately capture images of a patient and/or a ROI of the patient. In various embodiments, for example, the image capture device(s) 285 can focus on a ROI, zoom in on the ROI, center the ROI within a field of view by moving the image capture device(s) 285, or otherwise adjust the field of view to allow for better and/or more accurate tracking/measurement of the ROI.

The server 225 includes a processor 235 that is coupled to a memory 230. The processor 235 can store and recall data and applications in the memory 230. The processor 235 is also coupled to a transceiver 240. In some embodiments, the processor 235, and subsequently the server 225, can communicate with other devices, such as the computing device 210 through the connection 270.

The devices shown in the illustrative embodiment can be utilized in various ways. For example, either the connections 270 or 280 can be varied. Either of the connections 270 and 280 can be a hard-wired connection. A hard-wired connection can involve connecting the devices through a USB (universal serial bus) port, serial port, parallel port, or other type of wired connection that can facilitate the transfer of data and information between a processor of a device and a second processor of a second device. In another embodiment, either of the connections 270 and 280 can be a dock where one device can plug into another device. In other embodiments, either of the connections 270 and 280 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication can include near-field communications, such as passive radio-frequency identification (RFID) and active RFID technologies. RFID and similar near-field communications can allow the various devices to communicate in short range when they are placed proximate to one another. In yet another embodiment, the various devices can connect through an internet (or other network) connection. That is, either of the connections 270 and 280 can represent several different computing devices and network components that allow the various devices to communicate through the internet, either through a hard-wired or wireless connection. Either of the connections 270 and 280 can also be a combination of several modes of connection.

The configuration of the devices in FIG. 2 is merely one physical system 200 on which the disclosed embodiments can be executed. Other configurations of the devices shown can exist to practice the disclosed embodiments. Further, configurations of additional or fewer devices than the devices shown in FIG. 2 can exist to practice the disclosed embodiments. Additionally, the devices shown in FIG. 2 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, night vision cameras/detectors, thermal cameras, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

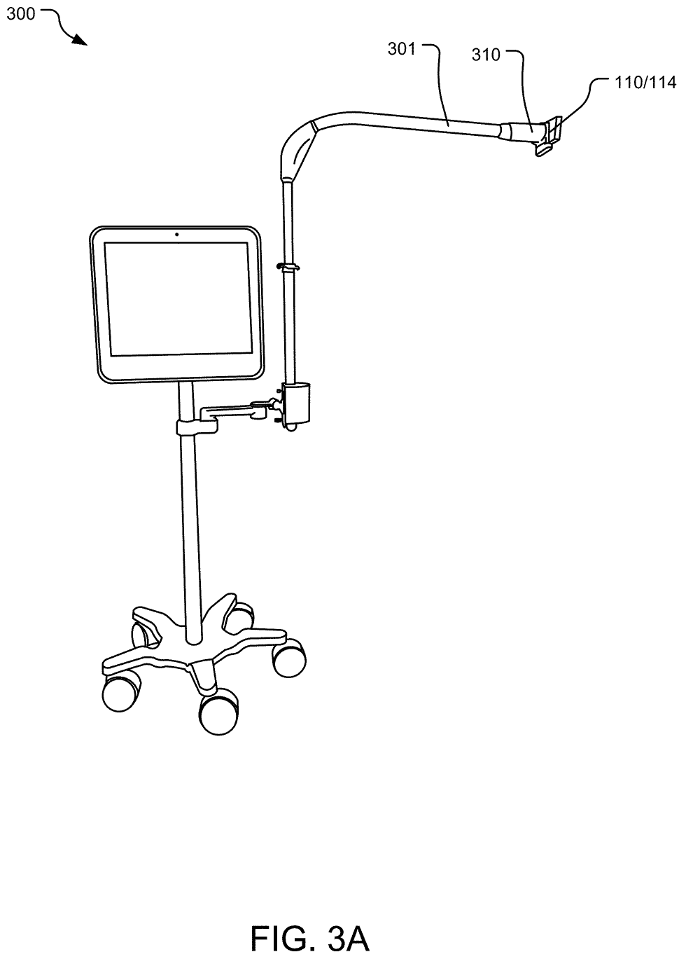

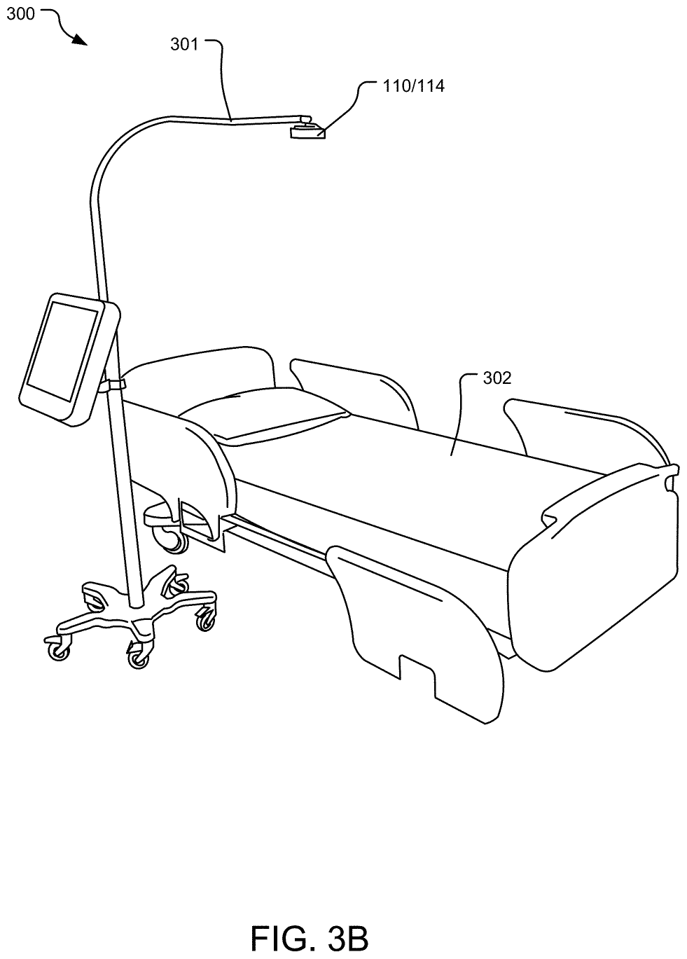

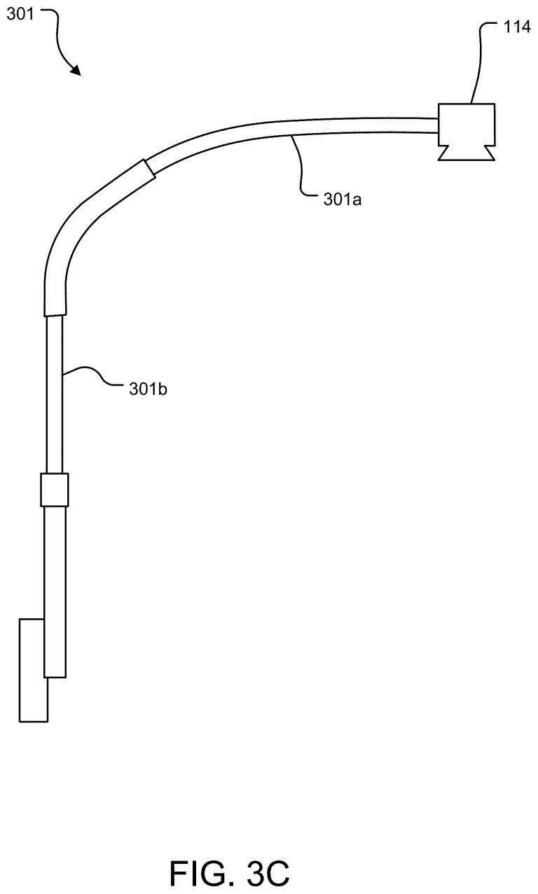

FIG. 1 generally shows non-contact detector 110 positioned over patient 112. While not shown in FIG. 1, in some embodiments, the non-contact detector 110 is moveable such that the detector can be positioned at various locations about patient 112. For example, as shown in FIG. 3A and FIG. 3B, the non-contact detector 110 (e.g., camera 114) may be attached to the distal end of a bendable arm 301 such that the location of camera 114 can be changed by manipulating the bendable arm 301. As shown in FIG. 3A, the proximate end of the bendable arm 301 can be connected to a pivot point such that the entirety of the bendable arm 301 may be pivoted about the pivot point to allow for further re-locating of the camera 114. In alternate embodiments, the bendable arm 301 is sufficiently long that a pivot point is not required and instead the camera 114 can be repositioned to any desirable location about the patient solely by manipulation of the bendable arm. FIG. 3A also shows the bendable arm 301 connected to a wheeled stand, which may allow for further re-positioning of the camera 114 through a combination of the bendable arm 301, the pivot point and the wheeled stand. Again, the bendable arm 301 may have sufficient length such that a wheeled stand is not required for moving the camera 114 to any desired location over the patient.

As shown in FIG. 3B, the bendable arm 301 extends over a patient bed 302 such that the camera 114 can be positioned over a patient lying in the patient bed 302. Due to any combination of the bendable arm 301, the pivot point to which the bendable arm 301 is attached, and the wheeled stand, the camera 114 can generally be moved such that is positioned over any portion of the patient. While the ability to move the camera 114 to any location around the patient can be beneficial to the clinician, it also allows the clinician to inadvertently position the camera 114 in such a way that diminishes the data collected by the camera 114, such as depth sensing data acquired when the camera 114 is a depth sensing camera. When the collected data is incomplete or inaccurate due to poor camera placement, subsequent patient parameters calculated from the depth sensing data are less reliable and accurate than when the camera 114 is properly positioned.

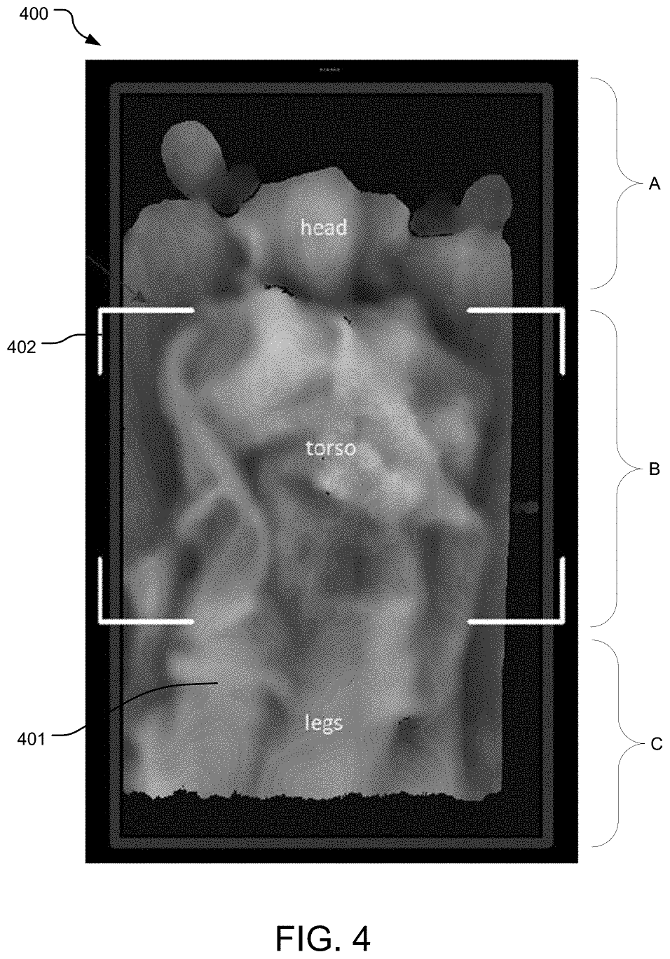

In order to aid in proper placement of the camera 114 to improve the quality and consistency of data collected by the camera 114, the systems and methods described herein may employ a target superimposed on the display used to display the imaging captured by the camera 114. With reference to FIG. 4, an embodiment of a display 400 broadcasting a real-time or near real-time video stream from camera 114 is shown. The display 400 includes the patient image 401 captured by the camera 114. In FIG. 4, the patient image 401 is a depth sensing image captured by camera 114 when camera 114 is a depth sensing camera. Superimposed on the patient image 401 displayed on display 400 is target 402. Target 402 is provided so that the clinician can adjust the positioning of the camera 114 until the target area of the patient (i.e., the portion of the patient from which data is to be collected) is located within the target 402. For example, in embodiments where the non-contact patient monitoring system is being used to obtain patient parameters related to patient breathing, the target area of the patient is generally the torso area of the patient. Accordingly, the clinician can manipulate the location of camera 114 until the patient's torso is located within the target 402 when viewing the video feed of camera 114 on display 400. By providing the clinician with the target 402 as a guide for us in proper placement of the camera 114, the accuracy and reliability of data obtained from the camera 114 is improved.

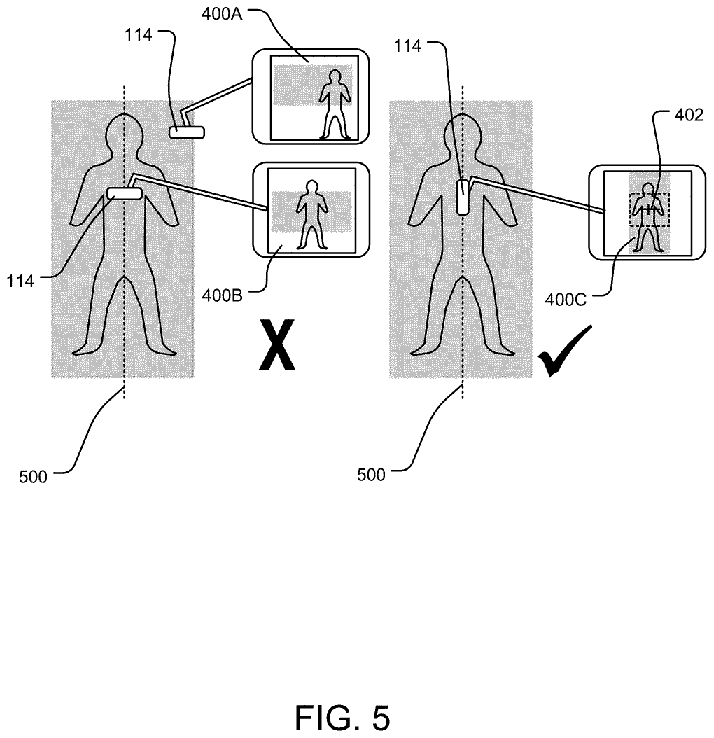

FIG. 5 provides an illustration of how the targeting aid 402 assists with proper placement of camera 114. Two improper camera placements are shown on the left side of FIG. 5. Displays 400a and 400b do not include target 402 and therefore the clinician is provided with no guidance for properly locating camera 114. In display 400a, the camera 114 is proximate the patient's head. Further, the camera 114 is oriented transverse to the longitudinal axis 500 of the patient. As such, display 400a (i.e., the grey shaded area) captures only the top half of the patient and the patient is off center in the display 400a. In display 400b, the camera 114 is centered on the patient, but the orientation of the camera 114 remains transverse to the longitudinal axis 500 of the patient. As a result, display 400b (i.e., the grey shaded area) excludes the patient's head and feet. In both instances, the quality of the data collected by the depth sensing camera will be reduced due to the improper placement of camera 114.

In contrast, the right side of FIG. 5 illustrates the embodiment where the display 400c includes target 402. The clinician is therefore able to reposition and reorient the camera 114 until the display 400c (i.e., the grey shaded area) includes the entirety of the patient's body and the patient's torso is located within target 402. When the clinician visually inspects the display 400c to confirm that the patient's torso is located within the target 402, this serves as confirmation that the camera 114 is properly positioned for accurate and reliable data collection.

Referring back to FIG. 4, the target 402 is generally shown as covering the middle segment of the patient image 401 displayed on display 400 (i.e., the segment labeled B in FIG. 4), which will generally correlate with the patient's torso when the camera is properly positioned. Segments A and C in FIG. 4, which may coincide with the patient's head and lower body, respectively, are excluded from the target 402. In some embodiments, segments A and B each cover roughly 30% of the image 401, while segment B covers approximately 40% of the image 401. However, it should be appreciated that the specific location of target 402, as well as the amount of the image 402 occupied by the target 402 is generally not limited. For example, the target may be located more toward the top or bottom of the image 401 then is shown FIG. 4, and the target can occupy more or less than 40% of the image.

The target 402 as shown in FIG. 4 also includes essentially the entire width of the patient image 401. However, it should be appreciated that the width of the target 402 is generally not limited, and may be less than the entire width of the image 401. Furthermore, the target need not be laterally centered on the display 400, but instead could be skewed to the left of right of the image 401.

The target 402 in FIG. 4 is defined by broken corner lines of a rectangular shape, using white as the color for the broken corner lines. However, it should be appreciated that the color, shape and/or design of the target 402 is not limited. In some embodiments, the shape of the target 402 may be oval, circular, square, or any of these shapes having rounded corners. The shape of target 402 may also be completely arbitrary and/or irregular, such as a shape generally corresponding to a human torso. In some embodiments, the target 402 is defined by solid lines, rather than the broken corner lines as shown in FIG. 4. Alternatively, broken lines extending across the entirety of the shape (rather than just in the corners) can also be used. Any color can be used for whatever shape and line type is used for target 402.

In other embodiments, the target 402 is displayed in the form of a shaded area, such that proper placement of the camera 114 is indicated when the patient torso (or other patient target area) is positioned within the shaded area. Alternatively, the non-target portion of the image 401 may be shaded, such that proper placement of the camera 114 is indicated when the patient torso (or other patient target area) is located in the unshaded area of the image 401.

In another embodiment a light projector may be attached next to the camera 114 on the bendable arm 301. The light projector can project a targeting pattern, similar in scale and size to target 402, onto the bed. The clinician can then manipulate the bendable arm 301 until the targeting pattern projected by the light projector is aligned with the target area of the patient's body, at which point the camera 114 is in the correct position for obtaining accurate and reliable data. In this method, the clinician need not look at or consult the monitor in order to properly align the camera 114. Once camera 114 is in the correct position, the light projector may be switched off.

In some embodiments, the software operated by the non-contact patient monitoring system may be designed such that the software automatically recognizes when the patient target area is located within the target and subsequently visually changes the target 402 when the patient target area is correctly positioned within the target 402 (or provides some other form of visual or audio indication of proper alignment). In such embodiments, the software includes one or more forms of recognition software capable of recognizing when the patient target area is located within the target area 402. For example, the software may be able to recognize the general shape of a human torso and therefore provides a visual and/or audio alarm when a torso shape is recognized within the target area 402. The recognition software may also use other parts of the patient to determine when a torso is located within the target area 402. For example, facial recognition software can be used to identify a patient's face in the image 401, and then determine a torso location based on the facial recognition and the probable location of the torso relative to the identified face. Once the torso is located in this manner, the software can provide a visual and/or audio indication when the identified torso is positioned within the target area 402. Any visual and/or audio feedback can be used. In some embodiments, the color of the target 402 changes from, e.g., red to green once the torso is identified as being located within the target area 402.

In some embodiments, the target 402 superimposed on the image 401 displayed on display 400 is stationary and does not change location, shape or size. In other embodiments, the location, shape and/or size of the target 402 can be dynamic. For example, once the camera 114 is initially located in the proper position such that the patient torso (or other patient target area) is located within the target 402, the target may “lock on” to the torso region. If the camera 114 is subsequently moved, the target 402 can change shape, size and or location on the display 400 to stay locked on to the previously identified torso. Such tracking may also be useful if the camera 114 remains in place, but the patient moves.

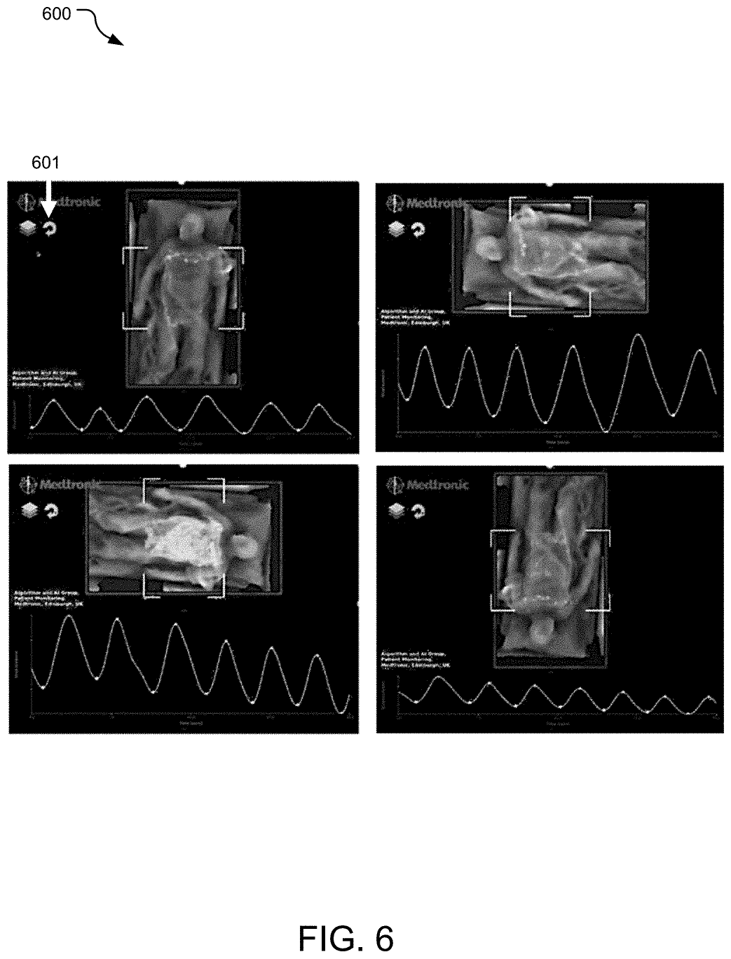

As shown in FIG. 4, the orientation of the patient image 401 on display 400 is oriented vertically such that the head of the patient is at the top of the display 400. In some embodiments, a user interface included on display 400 may include an option for rotating the orientation of the image 401. With reference to FIG. 6, a button 601 is provided that allows for rotating the patient image 401 90 degrees in a clockwise or counterclockwise direction. This feature may be advantageous in situations where the camera 114 is properly located and oriented (e.g., in parallel with the longitudinal axis of the patient), but the camera 114 is inverted such that the image transmitted to the display 400 presents the patient's head at the bottom of the display 400. This orientation can be easily changed by rotating the image 180 degrees using, e.g., button 601, and avoids the need for the clinician to instead further manipulate the camera 114 in order to get the desired image orientation. The ability to rotate the image also allows for horizontal orientation of the patient image 401 for clinicians that prefer a horizontal patient image orientation on display 400.

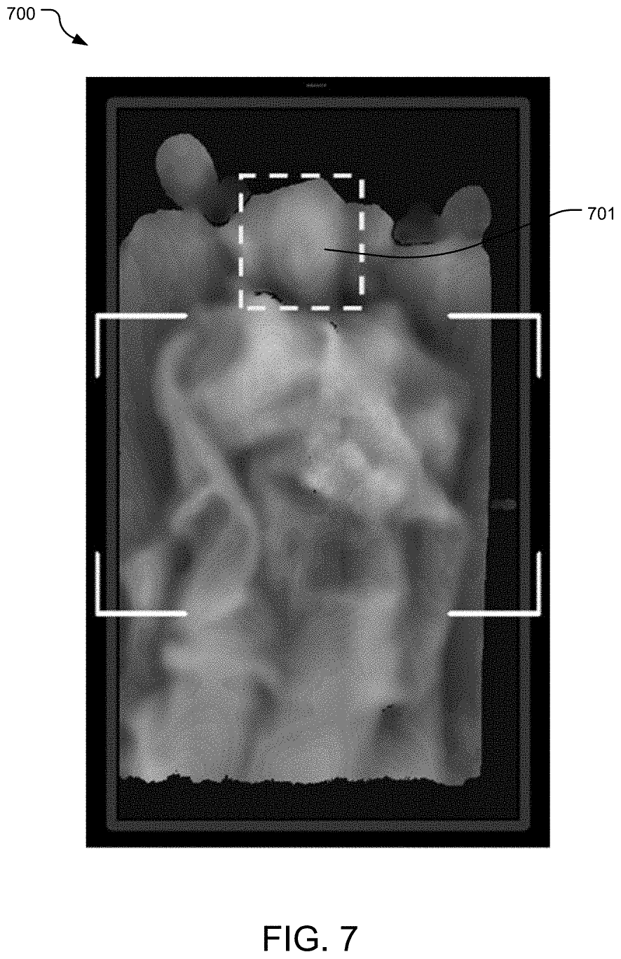

In some embodiments, the initial orientation of image 401 on display 400 may be automated using facial recognition software or other means. As shown in FIG. 7, facial recognition software is used to identify the patient face 701 in patient image 401. Once the face 701 is detected, image 401 is automatically oriented on display 400 such that the detected face 701 is at the top of the display 400. Any method of facial recognition can be used, including video streams of the patient other than depth sensing images. For example, a RGB stream, an IR stream, thermal imaging, or any other modality or combination of modalities can be used to locate the patient face using facial recognition software.

In addition to the location of camera 114 over the patient, the distance the camera 114 is placed away from the patient can also play a role in the accuracy and reliability of the data obtained from the camera 114. For example, in some embodiments, it is desirable for the camera to be 0.9 to 1.3 meters away from the patient. A camera 114 that is positioned too close or too far away from the patient may reduce the reliability and/or quality of the data obtained from the camera 114. Thus, in some embodiments, the systems and methods described herein further incorporate a means for providing the clinician with a distance measurement that measures the distance between the camera 114 and the patient. In some embodiments, this functionality may further include assisting and/or alerting the clinician to instances where the camera 114 is placed too far or too close to the patient, and/or to instances where the camera 114 is positioned at a desired distance from the patient to help ensure improved data collection.

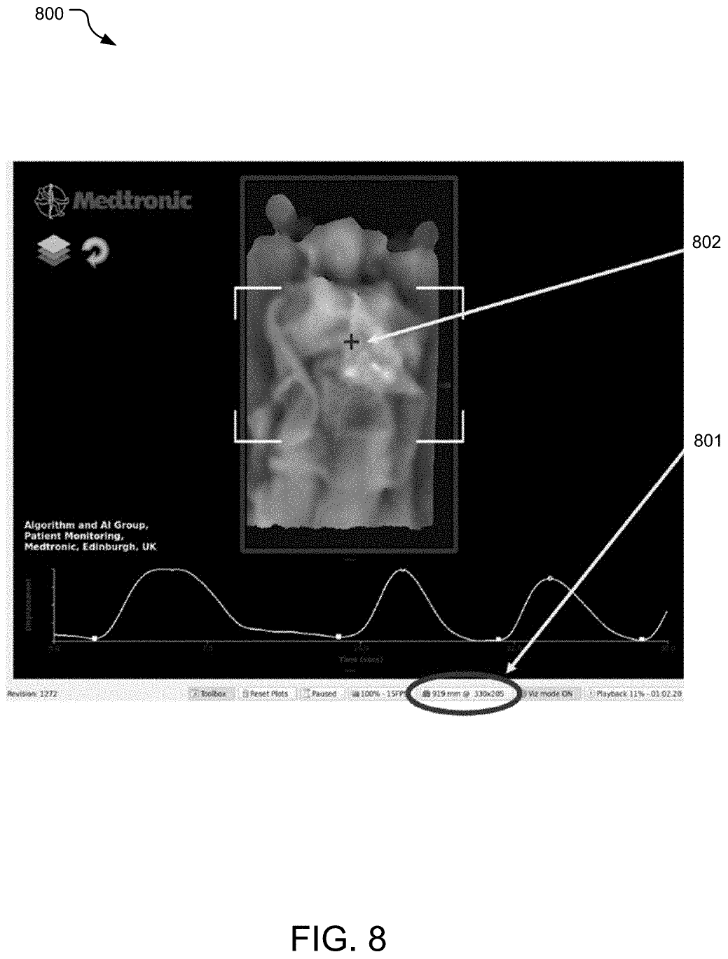

With reference to FIG. 8, the display 800 may include a distance reading 801. While shown in the bottom right hand corner of display 800, it should be appreciated that the distance reading 801 may be located anywhere on display 800. The display 800 and associated software may be configured such that a cursor 802 can be positioned over any part of the patient image to measure the distance between the camera 114 and the location on the patient designated by the cursor 802. This distance measurement 801 can then be used by the clinician to decide whether the camera 114 is at a good distance from the patient for reliable and accurate data collection, or whether the camera 114 needs to be moved further or closer towards the patient. Any manner of moving the cursor 802 about the display 800 can be used, such as through the use of a touch screen or a mouse.

In some embodiments, the ideal distance or range of distances is known or provided to the clinician for manual determination of whether the distance of the camera 114 away from the patient needs to be changed. That is to say, the clinician changes the distance of the camera 114 and re-checks the distance reading 801 until the clinician manually confirms that the distance reading 801 is within the known or provided distance range. In other embodiments, an ideal distance or range of distances is input into the system 100, and the display 800 and associated software provide automatic feedback regarding whether the camera 114 is at an ideal distance away from the patient or outside an ideal distance or distance range. The automatic feedback can be any type of audio and/or visual feedback. In some embodiments, the color of the distance reading 801 changes when the distance is either in the desired range or outside the desired range. For example, the distance reading 801 may be presented in green when the distance is within the desired range, and the color of the distance reading 801 may dynamically change as the camera distance is changed, such as dynamically changing from green to red when the camera 114 is moved outside the desired distance range.

As described previously with respect to FIG. 4, visual and/or audio feedback may also be provided to denote proper positioning and orientation of the camera 114, such as through the use of software to identify a patient target area (such as a patient torso) and then determine when the identified patient target area is properly positioned within the target 402. In some embodiments, the feedback provided for the distance of the camera 114 is combined with the feedback provided for the positioning/orientation of the camera 114. For example, the target 402 may be colored red until both the positioning/orientation of the camera 114 and the distance of the camera 114 are correct, at which point the target may change to a green color, indicating that the camera is in an optimal position for data collection. In some embodiments, if only one parameter is achieved (i.e., only correct camera position/orientation or only correct camera distance), the target 402 remains red until both parameters are met. In other embodiments, the color of target 402 may change from red to yellow when one parameter is met and from yellow to green when both parameters are met. Combining the feedback from both camera positioning/orientation and camera distance provides a relatively simple and streamlined process for assisting the clinician with proper location (both position and distance) of camera 114.

While the above description and FIG. 8 relate to the use of a moveable cursor to set a distance measurement, the target 402 may also be configured to include a fixed location within the target and from which is obtained the distance measurement. For example, a fixed crosshair can be provided in the center of target 402 such that the distance reading is always obtained from the center of the target 402, rather than requiring the clinician to manually set the cursor 802 before obtaining a distance measurement. The set location within the target 402 that distance is always measured from need not be in the center of the target 402, but can instead be located anywhere within target 402. The target may also include more than one fixed points for measuring distance. In such embodiments, the distance measured from each of the fixed points established within the target 402 may be averaged (including using weighted averages for each point, if desired), and the clinician may be provided with the averaged distance for determining if the camera is at an appropriate distance away from the patient.

With reference back to FIGS. 3A, 3B and 3C, the camera 114 may be attached to the distal end of a bendable/movable arm 301. Any type of bendable/movable arm 301 may be used provided that the arm 301 is capable of moving the camera 114 to different locations about the patient. The bendable arm 301 should also be able to hold and retain the camera 114 in whatever location the clinician moves it to. The length of the bendable arm 301 is generally not limited, but may preferably be of a length that allows for the camera 114 to be located at any location about the patient.

As shown in FIG. 3C, the bendable arm 301 may have a bendable component 301a and a fixed component 301b. In some embodiments, the fixed component 301b is aligned generally vertically. This vertical fixed component 301b may be telescoping to allow the bendable component 310a to be raised or lowered, and the fixed component 301b may be rotatable to allow for further re-positioning and movement of the bendable component 301a.

In some embodiments, camera 114 is connected to the distal end of the bendable arm 301 via a connection mechanism 310. The connection mechanism 310 is generally not limited provided that the connection mechanism 310 maintains a connection between the camera 114 and the bendable arm 301. The connection mechanism may allow for varying degrees of freedom of the camera 114 relative to the bendable arm. In some embodiments, the connection mechanism includes or incorporates a gimbal in order to allow for free movement of the bendable arm 301 but without altering a desired orientation of camera 114. For example, in some embodiments, it may be desirable that the camera 114 (and more specifically the lens or lenses of the camera 114) be fixedly oriented such that the camera/lens is always aligned parallel to the bed on which the patient is positioned. This camera/lens orientation may be desirable as a means for helping to ensure accurate and reliable data collection. For example, if the camera/lens is oriented at an angle with respect to the bed when directed at the patient, the depth sensing data obtained from the camera may be less reliable/accurate than if the camera/lens is positioned to be facing directly down on a patient (i.e., camera/lens aligned in parallel with the bed). By incorporating a gimbal as part of the connection mechanism 310, the camera 114 is generally able to be moved to any location about the patient (through manipulation of the bendable arm 301) but without changing the orientation of the camera/lens. That is to say, no matter where the camera 114 is located about the patient via movement of the bendable arm 301, the orientation of the camera 114 in a “parallel to the bed” position remains the same through the use of the gimbal.

While a gimbal component of connection mechanism 310 is generally described as being used to ensuring that the camera 114 remains oriented parallel to the bed on which the patient is disposed, it should be appreciated that the general, non-limiting, purpose of the gimbal may be to maintain a line of sight of the camera that is approximately orthogonal to the patient's chest, and that this orientation may require the camera and/or lens to be positioned differently from the above description depending on the specific camera and/or lens configuration. Regardless, the gimbal component may be used in any way necessary to help ensure this “line of sight orthogonal to patient's chest” alignment.

The gimbal may include the ability to lock and unlock the orientation of the camera 114. When in the locked position, the gimbal maintains the orientation of the camera 114 (e.g., in a “parallel to the horizon” orientation) regardless of where the camera is moved via manipulation of the bendable arm 301. When in the unlocked position, the gimbal may provide freedom of movement to the orientation of the camera such that it is not retained in a fixed “parallel to the horizon” orientation as the bendable arm is moved to change the position of the camera 114. The unlocked feature may provide flexibility for unique situations when the clinician requires the camera orientation to be something other than in the “parallel to the horizon” configuration.

In some embodiments, the bendable arm 301 and/or connection mechanism 310 may include one or more locking mechanism to lock the camera 114 in position once the camera 114 has been located where the patient target area is located within the target 402. Any suitable locking mechanism that prevents further movement of the bendable arm 301 and/or the connection mechanism 310 can be used. The ability to lock the camera 114 in position after it has been correctly located by the clinician may help to avoid situations where the camera is inadvertently bumped or moved after it has been correctly positioned. As such, this may help to ensure that the patient target area remains within the target 402. As noted previously, the software associated with the system 100 may include tracking technology such that the target 402 moves and/or changes shape or size if the patient moves. Such tracking technology may be specifically suitable for situations where the camera 114 is locked in place such that the patient target area remains within the target 402 even if a patient moves.

The connection mechanism 310 of the bendable arm 301 may also include actuators, for example a servo motor or any device that allows kinematic manipulation via a control input signal. The bendable arm 301 can then be manipulated so that the camera 114 is located at the optimum location to collect the most beneficial data. Manipulation of the bendable arm 301 can be performed automatically by analyzing the camera 114 images and subsequently adjusting the bendable arm 301 so that the most beneficial data can be collected.

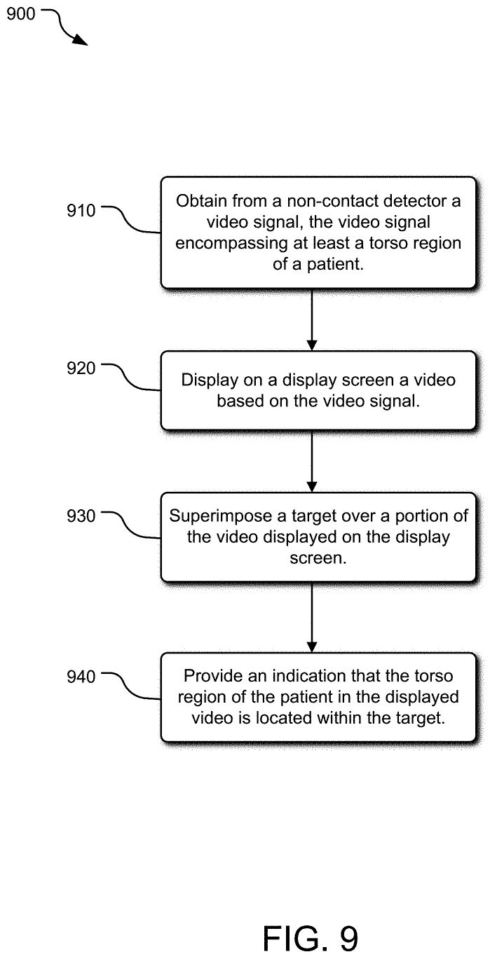

With reference to FIG. 9, a method 900 for assisting in the proper placement of a non-contact detector used in a video-based non-contact patient monitoring system may generally include a step 910 of obtaining from a non-contact detector a video signal, the video signal encompassing at least a torso region of a patient; a step 920 of displaying on a display screen a video based on the video signal; a step 930 of superimposing a target over a portion of the video displayed on the display screen; and a step 940 of providing an indication that the torso region of the patient in the displayed video is located within the target. As discussed in greater detail previously, steps 910 and 920 generally related to the use of the system 100 shown in FIG. 1, wherein the non-contact detector 110 (such as camera 114) provides a video signal of the patient, and a video of the patient based on the video signal is displayed on display screen 122. Step 930 generally relates to the target 402 shown in FIG. 4, and more specifically how the target 402 is superimposed over a portion of the video displayed on the display screen. In step 940, the methods described herein generally work to provide an indication or alert to the clinician to let the clinician know when the camera 114 has been properly positioned to best ensure the collection of accurate and reliable data from the camera. As discussed in greater detail previously, such an indication is typically provided to the clinician when the camera 114 has been positioned such that the target 402 is encompassing the torso region of the patient as displayed on the display screen. As also described in greater detail previously, the indication or alert may be provided in the form of a visual and/or audio alert, such as the target 402 changing color (e.g., from red to green) when the camera 114 is properly positioned.

Various previously described embodiments generally relate to systems and methods wherein a screen or display is visible to the clinician when moving the camera such that the clinician can use the screen or display to confirm when the camera is properly positioned. That is to say, the screen or display is located sufficiently local to the camera that while the clinician manipulates the camera, the clinician can view and use the display to confirm when the camera is properly positioned (e.g., when the patient's torso as displayed on the screen is located within a target superimposed on the screen of the display). This configuration is shown in, e.g., FIGS. 3A and 3B, wherein a screen or display is mounted directly to a portion of the bendable arm 301 having a camera 114 attached to a distal end thereof. However, it should be appreciated that in some configurations, a screen or display proximate the camera 114 will not be available. For example, a screen configured to display the image obtained from the camera 114 may be located outside of the room in which the camera 114 and patient being monitored are located, such as at a central monitoring location (e.g., nurse's station). In such embodiments, the clinician is not able to rely on the display to confirm proper positioning of the camera 114, and therefore other features must be provided to assist with proper camera placement.

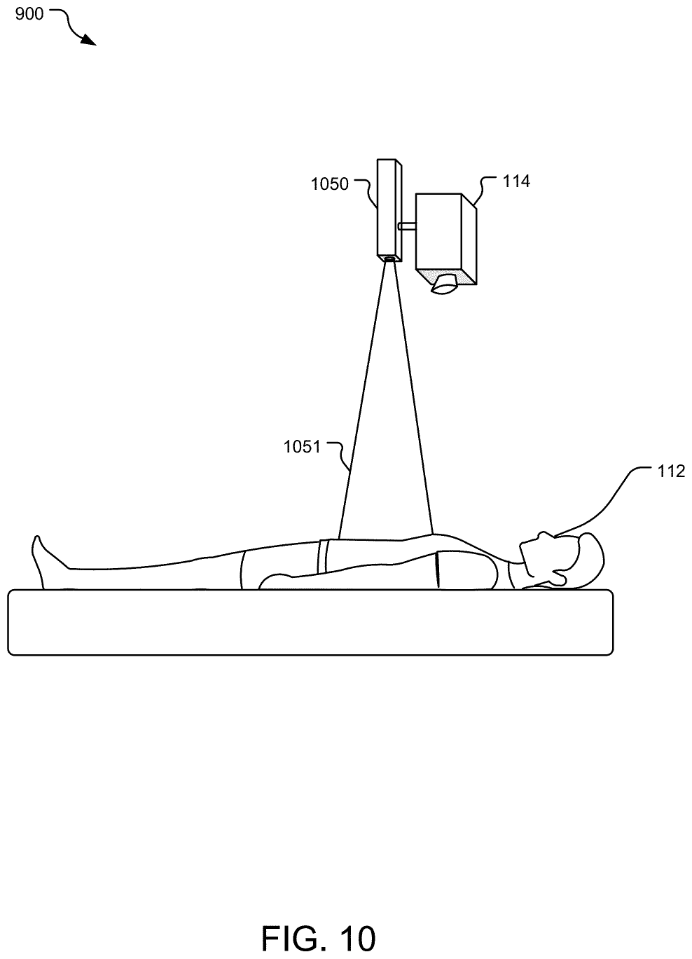

In some embodiments, systems and methods wherein a display is not locally available include a projector configured to project a target on to an object (e.g., the patient, the patient's bed, etc.) located within the camera's field of view. This configuration is generally shown in FIG. 10. As previously described, camera 114 is moveable such that camera 114 can be positioned such that the patient 112 or a portion of patient 112 is within the field of view of the camera 112. A projector 1050 is mounted on or otherwise secured to or with the camera 114, with the lens of the projector 1050 facing generally in the same direction as the lens of the camera 114. In this manner, the projector 1050 is capable of projecting on to the patient 112 a target 1051, with the target 1051 being within the field of view of the camera 114.

In some embodiments, the manner in which the projector 1050 is secured to or with the camera 114 and otherwise calibrated is such that when a targeted area of the patient 112 (e.g., the patient's torso) is located within the projected target 1051, the camera 114 is in a position capable of obtaining depth data suitable for use in calculating a patient breathing parameter. Calibration of the positioning and/or alignment of the projector 1050 connected to the camera 114 can be carried out in a suitable way, including by setting the camera 114 in the desired position where sufficient depth data can be obtained, and then manipulating the positioning and/or other settings of the projector until the target 1050 is appropriately aligned (e.g., encompasses the desired portion of the patient 112). Following calibration, subsequent use of the camera 114 and projector 1050 should ensure that when the projected target 1051 encompasses the targeted portion of the patient 112, the camera 114 will be in a position to acquire the required depth sensing data for determining a patient breathing parameter.

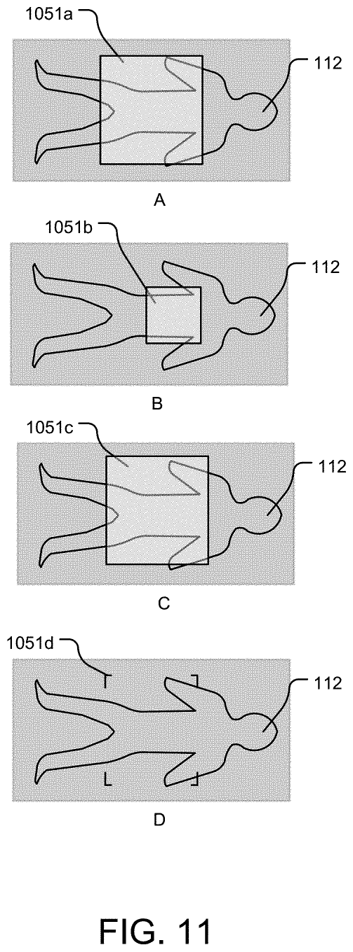

The specific visual scheme or appearance of the target 1051 projected by projector 1050 is generally not limited. That is to say, the shape, size, color, etc., of the target 1051 may be of any desired visual appearance. FIG. 11 illustrates four exemplary, though non-limiting, targets 1051a through 1051d. In one embodiment (image A of FIG. 11), target 1051a projected on patient 112 has a relatively large rectangular shape whose entire interior is colored yellow. In another embodiment (image B of FIG. 11), target 1051b is similar in shape, color and fill to target 1051a shown in image A, but the size of the rectangle is smaller, which can allow for targeting of more localized regions of the patient 112. In another embodiment (image C of FIG. 11), target 1051c is a similar shape and size to target 1051a shown in image A, but does not include a colored interior. Instead, the target 1051c uses a yellow outline to denote the boundaries of the target. In another embodiment (image D of FIG. 11), target 1051d is a similar shape and size to target 1051a shown in image A, but does not include a colored interior or a boundary line extending around the entirety of the shape. Instead, the target 1051d uses yellow corner segments to denote the boundaries of the target.

While FIG. 11 provides some examples of the visual representation of target 1051 as projected by projector 1050, other visual representations of the target 1051 can be used. For example, the shape of the target 1051 may be a circle, ellipses, triangle, or any regular or irregular shape. In some embodiments where an irregular shape is used, the shape of the target 1051 is the outline of a human torso, which can further aid in positioning the camera (i.e., the camera is properly positioned when the torso-shaped target is aligned with the torso of the patient 112). As noted previously with respect to FIG. 11, the target 1050, regardless of shape and size, can have a colored interior portion or a clear interior portion, in which case the outline of the shape denotes the target 1051. When the interior of the shape is colored, the coloring may be a solid coloring or a patterned coloring. When an outline is used for target 1051, the outline can be solid lines, dashed lines, corners segments only, or any other pattern. The specific color used for the target 1051 is also not limited.

The projector 1050 may be configured, in connection with other components of the system 100 previously described (e.g., computing device 115, processor 118, etc.) such that the projector 1050 only projects target 1051 at certain times and/or under certain conditions. In a simple configuration, the projector 1050 projects target 1051 when a switch associated with the projector 1050 is turned on. The switch can be, for example, a piece of hardware that is depressed, turned or otherwise maneuvered, or an icon on a touch screen associated with the projector 1050 or some other component of system 100. In some embodiments, the projector 1050 and/or an associated component of system 100 that is communicatively coupled with the projector 1050 includes a timer such that when the target 1051 is turned on, the target 1051 remains on for a predetermined period of time monitored by the timer. For example, once turned on, the target 1051 may remain on for a predetermined period of time (e.g., 30 second, 60 seconds, 90 seconds, etc.), after which the projector 1050 is automatically turned off. The predetermined period of time that the projector 1050 remains on may be selected based on the amount of time typically needed for a clinician to correctly position the camera 114 over the patient 112. In other embodiments, the projector 1050 remains on until the manually turns off the projector 1050.

In some embodiments, a switch or other manual means is not used to turn on the projector 1050, but instead the projector 1050 is configured to automatically turn on when motion of the camera 114 to which the projector 1050 is attached, or motion of the projector 1050 itself, is detected. Motion of the projector 1050 and/or camera 114 can be determined using any suitable means. In some embodiments, the projector 1050 and/or camera 114 is equipped with an accelerometer, and motion detected by the accelerometer results in the projector 1050 being automatically turned on. In other embodiments, a sufficient change in the depth data being collected by the camera 114 initiates the projector 1050. For example, if the camera 114 collects depth data indicating that the camera is 1.1 meters away from the patient 112 and this measurement remains constant over a period of time, then the projector 1050 remains off. However, once the camera 114 begins to collect different depth data (e.g., that the camera is now 1.5 meters away from the patient), the assumption is that the camera 114 is being moved, and therefore the projector 1050 is automatically turned on to assist the clinician in accurately and correctly positioning the camera 114.

Regardless of the manner in which the projector 1050 is automatically turned on (e.g., via accelerometer, via change in depth reading, etc.), the projector 1050 can be programmed to automatically turn off after a predetermined period of time as described previously. Alternatively, the projector 1050 can be programmed so that is remains on until motion of the camera 114 and/or projector 1050 has ceased for longer than a predetermined period of time. For example, an accelerometer associated with the camera 114 and/or projector 1050 can detect motion, at which point the projector 1050 is automatically turned on. The accelerometer can continue to sense motion for the next 3 minutes, and as a result, the projector 1050 remains on. However, once the accelerometer senses no movement for longer than a predetermined period of time (e.g., 15 seconds, 30 second, etc.), the projector 1050 can be automatically turned off. A similar manner of turning off the projector 1050 can be used when changes in depth measurements are used to detect motion of the camera 114. In such embodiments, once the camera 114 determines that the depth measurement has stopped changing for longer than a predetermined period of time (and after previously detecting changes in depth data such that the projector 1050 has been turned on) the projector 1050 can be automatically turned off.

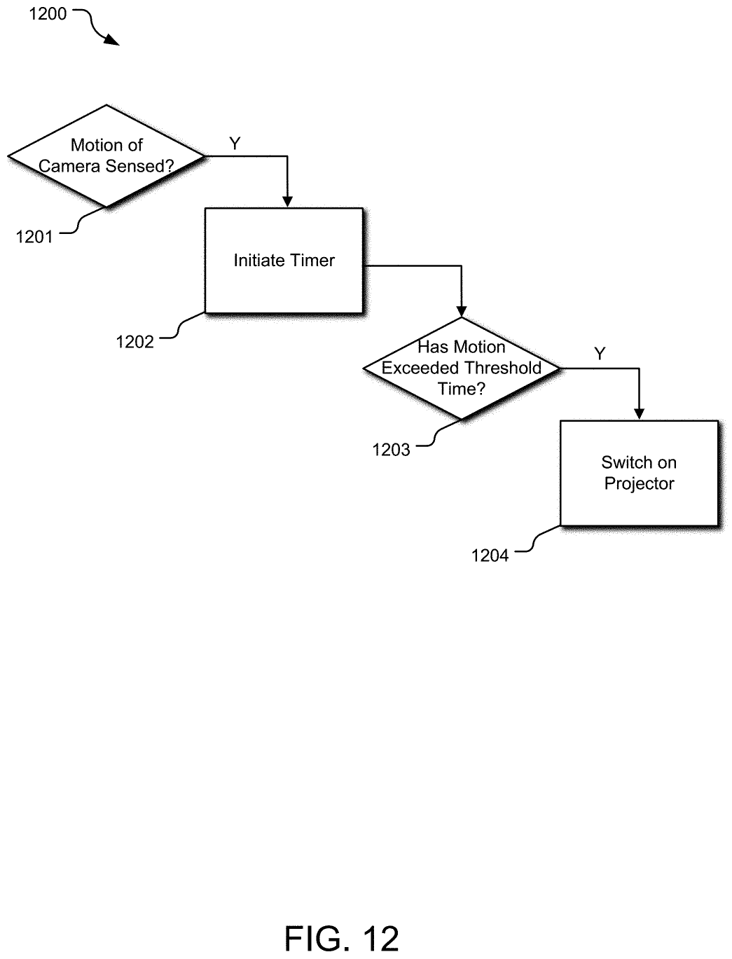

FIG. 12 illustrates an exemplary method 1200 for automatically turning the projector 1050 on when motion of the camera 114 is detected. At step 1201, it is determined whether motion of the camera 114 is detected. Detecting motion of the camera 114 can be by any means, including those described previously (e.g., via use of an accelerometer). If motion is not detected, then no action is taken at step 1201. If motion is detected at step 1201, then a timer may be initiated at step 1202. Because camera 114, projector 1050, and means for determining motion (e.g., accelerometer) are communicatively coupled with system 100, the detection of motion may be communicated to, for example, the computing device 115 of the system 100, which may then initiate the timer. At step 1203, it is determined whether the detected motion persists for longer than a predetermined period of time monitored by the time (e.g., longer than 3 seconds, longer than 5 seconds, longer than 10 seconds, etc.). Step 1203 helps to ensure that any minor motion of the camera of minimum duration, such as when the camera is inadvertently and only momentarily bumped by a clinician, does not initiate the projector 1050. Instead, the motion must be prolonged, thus serving as a better indication of the camera 114 being moved, and hence the need for the projector 1050 to be turned on to aid in positioning the camera 114. If motion is detected for longer than the predetermined period of time at step 1203, then method 1200 proceeds to step 1204, in which the projector 1050 is automatically turned on to thereby project target 1051.

As discussed previously, target 1051 may generally be in the form of a regular or irregular shape of any size and/or color. In some embodiments, the projector further projects, such as part of target 1051, additional images to provide the clinician with additional information. In some embodiments, the additional information projected by projector 1050 relates to the distance between the camera 114 and the patient 112. For example, the projector 1050 may also project on to the patient 112 (either inside of or separate from target 1051) a number indicating the distance between the camera 114 and the patient 112. Projection of this distance number may aid the clinician in ensuring that the camera 114 is positioned a desirable distance away from the patient 112. For example, it may be desirable that the camera 114 is positioned about 1.1 meters away from the patient 112 to ensure collection of reliable data to be used in determining a patient breathing parameter, and therefore the clinician may move the camera 114 closer or further away from patient 112 until the projected depth reading is at or close to 1.1 meters. The projected distance may change in real time or near real time as the camera 114 is moved.

Other manners for visually representing that the camera 114 is positioned a desired distance from the patient 112 can also be used. For example, a check mark can be projected (inside or outside of target 1051) when the distance between the camera 114 and the patient 112 is determined to be within a desirable range (e.g., between 1.0 and 1.2 meters). The projector 1050 may also add text (inside or outside of targe 1051), such as “Good Alignment”, when the camera 114 is located a distance away from patient 112 that is within the desired range. In other embodiments, the visual representation of the target 1051 is changed when the camera 114 is determined to be a distance away from the patient 112 that falls within a desired range. In such embodiments, the target 1051 may include at least a first visual scheme and a second visual scheme. The projector 1050 projects the target 1051 using the first visual scheme when the camera 114 is outside of the desired distance range. Once the camera 114 is moved to a distance within the desired range, the projector 1050 changes the projection of target 1051 such that the second visual scheme is used, and which thereby denotes to the clinician that camera 114 is at an acceptable distance away from the patient 112.

The change from the first visual scheme to the second visual scheme for target 1051 can be any desired change in visual scheme. In some embodiments, the first visual scheme uses a red color scheme to thereby denote the distance of camera 114 from patient 112 is outside of the desired range, and the second visual scheme uses a green color scheme to thereby denote the distance of camera 114 from patient 112 is within the desired range. Other changes from the first visual scheme may include changes in the shape or size of target 1051, or any combination thereof.

In addition to or as an alternative to changing the visual scheme of target 1051 when camera 114 is within the desired range of distances, the camera and/or projector may include a screen, display, light, or other indicator to help indicate when camera 114 is within a desired range of distance from patient 112. For example, a small screen or display may be associated with either the camera 114 or the projector 1050, and the screen or display may be used to indicate when the distance of the camera 114 from the patient 112 is within a desired range. In another embodiment, one or more lights may be associated with the camera 114 and/or projector 1050, and the light may be used to indicate when the camera 114 is a desired distance away from the patient 112. In such embodiments, the light may turn from off to on to denote correct distance, or may change from one color (e.g., red) to another color (e.g., green).

The specific distance used to determine alignment of camera 114 is generally not limited. In some embodiments, the measurement used to determine when the distance between the camera 114 and the patient 112 is within a desired range is the distance from the camera to a center point of the target 1051. In another embodiment, the measurement used to determine when the distance between the camera 114 and the patient 112 is within a desired range is the average of all distance data points, or some subset of distance data points, within the field of view of the camera 114.

Once proper alignment of camera 114 has been established (both in terms of distance of camera 114 from patient 112 and the positioning of camera 114 such that the desired portion of patient 112 is within target 1051), system 100 can be configured to store an image of the patient 112. The stored image may then become a reference image used to periodically or continuously check whether the camera 114 remains in proper alignment. If system 100 detects a difference between the reference image and the current image that exceeds a threshold value, then the system 100 may initiate an alarm intended to indicate to the clinician that the camera 114 is no longer in good alignment with the patient 112. Camera 114 may fall out of good alignment for any of a variety of reasons, including, for example, inadvertent movement of the camera 114, movement of the patient 112, movement of the patient's bed, etc. Any type of alert may be triggered in this scenario, including a visual alert, an audio alert, etc., including any combination of alerts. Visual alerts may be displayed as part of the target 1051, on a separate display screen associated with system 100, on the projector 1050 and/or camera 114, etc. For example, a screen, display, or light associated with the camera 114 or projector 1050 as described previously may be used to display a visual alert. Such visual alert could include a screen, display or light turning red, or a screen, display or light flashing.

For the sake of simplicity and example, the technology described herein has been disclosed with respect to the use of the systems and methods for monitoring patient breathing parameters, and therefore has focused primarily on instances where a targeting aid is used to ensure a camera is properly positioned to view a patient's torso. However, it should be appreciated that embodiments and aspects described herein are equally applicable to monitoring other patient parameters and/or other portions of a patient's body. For example, the systems and method described herein are equally applicable to using a target to ensure that a camera is properly placed to be aimed at a patient's head for collecting data pertaining to the patient's head. In such examples, the systems and methods may be used for monitoring patient temperature, in which case the camera is a temperature sensing camera, and the target is used to ensure that the camera is focused on portions of the patient's head from which reliable and accurate temperature information can be obtained. Numerous other examples for other patient body segments and patient parameters apply.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A video-based patient monitoring method, comprising:
obtaining from a non-contact detector a video signal, the video signal encompassing at least a torso region of a patient;
displaying on a display screen a video based on the video signal;
superimposing a target over a portion of the video displayed on the display screen; and
providing an indication that the torso region of the patient in the displayed video is located within the target.

2. The method of claim 1, wherein the non-contact detector is a video camera.

3. The method of claim 2, wherein the video camera is a depth-sensing video camera.

4. The method of claim 1, wherein the target superimposed over a portion of the video displayed on the display screen is vertically centered on the video displayed on the display screen.

5. The method of claim 1, wherein the target comprises a geometric shape defined by solid lines, broken lines or broken corner lines.

6. The method of claim 1, wherein the target comprises a shaded area.

7. The method of claim 1, wherein the target comprises an unshaded area and the non-target area of the video is shaded.

8. The method of claim 1, further comprising:
automatically identifying when the torso region of the patient is positioned within the target; and
visually changing the appearance of the target when the torso region of the patient is positioned within the target, providing an audible sound when the torso region of the patient is positioned within the target, or both.

9. The method of claim 8, further comprising:
automatically identifying when a vertical distance between the patient and the non-contact detector falls within a predetermined range.

10. The method of claim 9, wherein visually changing the appearance of the target when the torso region of the patient is positioned within the target, providing an audible sound when the torso region of the patient is positioned within the target, or both only occurs if the vertical distance between the patient and the non-contact detector falls within the predetermined range.

11. The method of claim 1, further comprising prompting a user to align the target with the torso region of the patient.

12. A video-based patient monitoring method, comprising:
displaying on a display screen a patient image based on a video signal obtained from a video camera, the patient image having superimposed thereon a target encompassing a portion of the patient image; and
manipulating a bendable arm to which the video camera is attached to reposition the video camera until a patient target area in the patient image is located within the target on the display screen;
wherein a connection between the bendable arm and the video camera includes a gimbal, the gimbal being configured to maintain the orientation of the video camera in a position generally parallel to a bed on which the patient is positioned during manipulation of the bendable arm.

13. The method of claim 12, wherein the video camera is a depth-sensing video camera.

14. The method of claim 12, further comprising:
during manipulating the bendable arm, automatically identifying when the patient target area is positioned within the target; and
visually changing the appearance of the target when the patient target area is positioned within the target, providing an audible sound when the patient target area is positioned within the target, or both.

15. The method of claim 14, further comprising:
during manipulating the bendable arm, automatically identifying when a vertical distance between the patient and the video camera falls within a predetermined range.

16. The method of claim 15, wherein visually changing the appearance of the target when the patient target area is positioned within the target, providing an audible sound when the patient target area is positioned within the target, or both only occurs if the vertical distance between the patient and the non-contact detector falls within the predetermined range.

17. A video-based patient monitoring system, comprising:
a video camera configured to obtain a video signal;
a bendable arm attached at a distal end to the video camera; and
a display, the display configured to:
display a patient image based on the video signal; and
superimpose over the patient image a target;
wherein the video camera is moveable about a patient via manipulation of the bendable arm; and
wherein the system is configured to:
automatically determine when a target patient area of the patient image is located within the target superimposed on the patient image via manipulation of the bendable arm and corresponding repositioning of the video camera; and
provide visual and/or audible feedback when the system automatically determines that the target patient area of the patient image is located within the target superimposed on the patient image.

19. The system of claim 18, wherein the system provides visual feedback, and the visual feedback comprises the color of the target changing from a first color to a second color when the target patient area of the patient image is located within the target superimposed on the patient image.

20. The system of claim 18, wherein the system further comprises:
an attachment mechanism configured to attach the video camera to a distal end of the bendable arm, the attachment mechanism comprising at least a gimbal configured to maintain the video camera in an orientation generally parallel to a bed on which the patient is positioned during manipulating of the bendable arm.
